(19) United States
(12) Patent Application Publication
(10) Pub. No.: US 2026/0020750 A1
(43) Pub. Date: Jan. 22, 2026

(54) Dual View Endoscope

(71) Applicant: OmniVision Technologies, Inc., Santa Clara, CA (US)

(72) Inventors: Chang-Long Chen, Taoyuan City (TW); Jau-Jan Deng, Taipei (TW)

(21) Appl. No.: 18/778,418

(22) Filed: Jul. 19, 2024

(51) Int. Cl. A61B 1/00

(52) U.S. Cl. CPC A61B 1/00181; A61B 1/00179

(57) ABSTRACT

A dual view endoscope includes an image sensor and a functional module disposed in front of the image sensor, the functional module having at least the first lens and the second lens attached to the first lens. The second lens located between the first lens and the functional module. The first lens is configured to expand field of view of said image sensor, while the second lens is configured to collect light rays from lateral directions and deflect the light rays into the image sensor. The image sensor and functional module are aligned with an axial direction of the dual view endoscope.

### TECHNICAL FIELD

The present invention relates to an endoscope, and more particularly, a dual view endoscope.

### BACKGROUND

An endoscope is a medical device with a light source attached, which is used to inspect inside a body cavity or organ through a body orifice. The endoscope is typically a long flexible tube with a lens at one end and a video camera at the other end, where the end with the lens is inserted into the patient.

Many conventional endoscopes include a forward viewing fiberoptic bundle or camera disposed at the distal end of the endoscope that captures and transmits visual images to an eyepiece, in the case of fiberoptic bundle, or to a video display monitor at the proximal end. Visual images collected by the fiberoptic bundle or camera are then transmitted back to the physician through a series of lenses extending through the endoscope barrel from a field lens at the distal end to the eyepiece lens assembly at the endoscope's proximal end. The field lens commonly faces in a forward or longitudinal direction, although a forward-oblique orientation is often provided.

A conventional forward-viewing endoscope 100, as illustrated in FIG. 1A, has only single view and limited forward field of view (FOV). When the conventional forward-viewing endoscope 100 being used in some special environments or scenarios, for example in an internal cavity 102 having a bulge structure 103 on its sidewall 104, an object of interest 106 formed on the bulge structure 103 can't be detected due to the image of the object of interest 106 might be blocked. As shown in FIG. 1B, only the image of the bulge structure 103 is captured by the image sensor 108 but the image of the object of interest 106 can't be detected. This situation can also happen in an environment such as the lower gastro-interest tract, flexures, tissue folds, and unusual geometries of the organ, which may prevent the endoscope's forward-looking camera from viewing behind tissue folds, flexures, and other hidden areas of the lumen.

To solve the aforementioned issues faced by the conventional forward-viewing endoscope 100 depicted in FIG. 1A, an oblique-viewing endoscope 200 with a prism 206b was proposed to collect light rays 202 at an oblique angle with respect to its optical axis 204. The oblique-viewing endoscope 200 includes a deflection lens group 206 attached to an imaging module 208, as illustrated in FIG. 2A, the deflection lens group 206 having a negative lens 206a and a prism 206b. The negative lens 206a is used to collect light rays 202 from lateral directions. The prism 206b deflects and reflects the light rays 202 into the image module 208. As shown in FIG. 2A, the prism 206b has the first reflective surface 206-1 and the second reflective surface 206-2, where the first reflective surface 206-1 is coated with reflective material and the second reflective surface 206-2 is a total reflection surface. By the arrangement of deflection lens group 206 together with the image module 208, the light rays 202 coming from various oblique angles with respect to the optical axis 204 of the endoscope 200 can be collected and deflected into the image module 208. As shown in FIG. 2B, the image of the object of interest 210 formed on the bulge structure 212 can be captured by the image sensor 208a.

Some drawbacks are still existed in the oblique-viewing endoscope 200 shown in FIG. 2A, although it can resolve the visual blind-spot issue caused by the bulge structure 212 of the internal cavity 214. One disadvantage is that the prism 206b is difficult to be made by molding or lens replication and the alignment tolerance of the prism 206b is relatively tight. The other disadvantage is that only one side of the internal cavity 214 can be imaged and inspected at a time while using the oblique-viewing endoscope 200. To capture an image from different sides of the internal cavity 214, the endoscope 200 must be maneuvered, repositioned, or move back and forth. All these maneuvers of the endoscope 200 prolong the procedure and cause additional discomfort to the patient.

According to the aforementioned drawbacks of the disclosed conventional endoscopes, a better solution to overcome these issues is therefore needed.

### SUMMARY OF THE INVENTION

In one aspect, the present invention provides a dual view endoscope, which includes an image sensor and a functional module disposed in front of the image sensor, the functional module includes at least the first lens and the second lens attached to the first lens. The first lens is a meniscus lens having two surfaces, one surface has planar shape (planar surface) and the other surface has spherical or aspherical shape (spherical or aspherical surface). The second lens is a freeform lens having two surfaces, one surface has planar shape (planar surface) and the other surface has freeform shape (freeform surface). The freeform lens is located between the meniscus lens and the image sensor. The meniscus lens is attached to the freeform lens by placing its spherical or aspherical surface adjacent to the freeform surface of the freeform lens. The first lens is configured to expand field of view of the image sensor, while the second lens is configured to collect light rays from lateral directions and deflect the light rays into the image sensor. The image sensor and functional module are aligned with an axial direction of the dual view endoscope.

In another aspect, the present invention provides a lens module for endoscope, which comprises the first lens and the second lens attached to the first lens. The second lens is located between the first lens and the endoscope. The first lens is configured to expand field of view of the endoscope. The second lens is configured to collect light rays from lateral directions and deflect the light rays onto an image sensor of the endoscope.

### BRIEF DESCRIPTION OF THE DRAWINGS

The components, characteristics and advantages of the present invention may be understood by the detailed descriptions of the preferred embodiments outlined in the specification and the drawings attached:

FIG. 1A illustrates a schematic drawing of a conventional forward-viewing endoscope inspecting an internal cavity with a bulge structure and an object of interest formed on the bulge structure according to one prior art.

FIG. 1B illustrates the image of the bulge structure of the internal cavity captured by an image sensor of the forward-viewing endoscope shown in FIG. 1A.

FIG. 2A illustrates a schematic drawing of a conventional oblique-viewing endoscope inspecting an internal cavity with a bulge structure and an object of interest formed on the bulge structure according to another prior art.

FIG. 2B illustrates the image of the bulge structure of the internal cavity together with the object of interest formed on the bulge structure captured by an image sensor of the oblique-viewing endoscope shown in FIG. 2A.

FIG. 3A illustrates a schematic drawing showing a proposed dual view endoscope inspecting an internal cavity with a bulge structure and interest particles formed on the bulge structure according to one embodiment of the present invention.

FIG. 3B illustrates the image captured by the image sensor, including distal scene from the forward field of view and the interest particles formed on the bulge structure shown in FIG. 3A.

FIG. 4A illustrates a functional module coupled to an image module according to one embodiment the present invention.

FIG. 4B illustrates a functional module coupled to an image module according to other embodiment of the present invention.

FIG. 4C illustrates a functional module coupled to an image module according to another embodiment of the present invention.

FIG. 5A is a schematic cross-sectional illustration of light rays passing through a meniscus lens according to one embodiment of the present invention.

FIGS. 5B-5D illustrate various meniscus lens embodiments according to the present invention.

FIGS. 6A-6C are schematic cross-sectional illustrations of light rays passing through a freeform lens according to various embodiments of the present invention.

FIGS. 6D-6E illustrate various freeform lens embodiments according to the present invention.

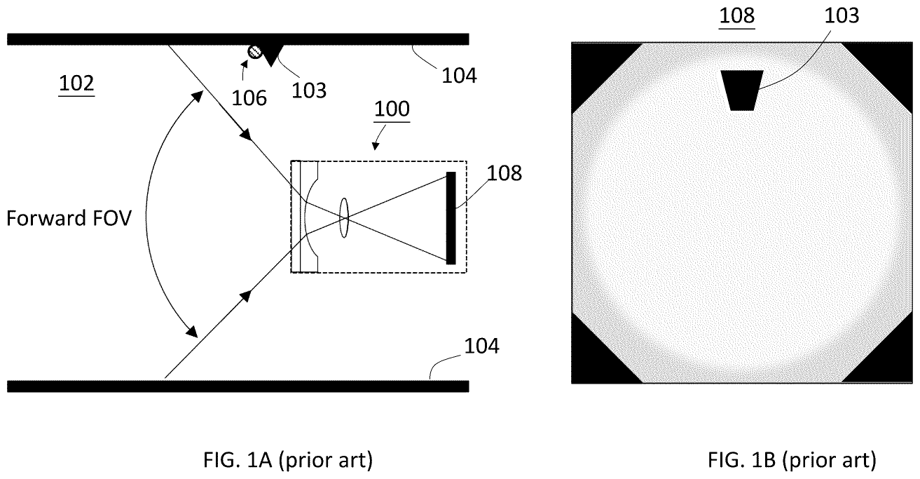

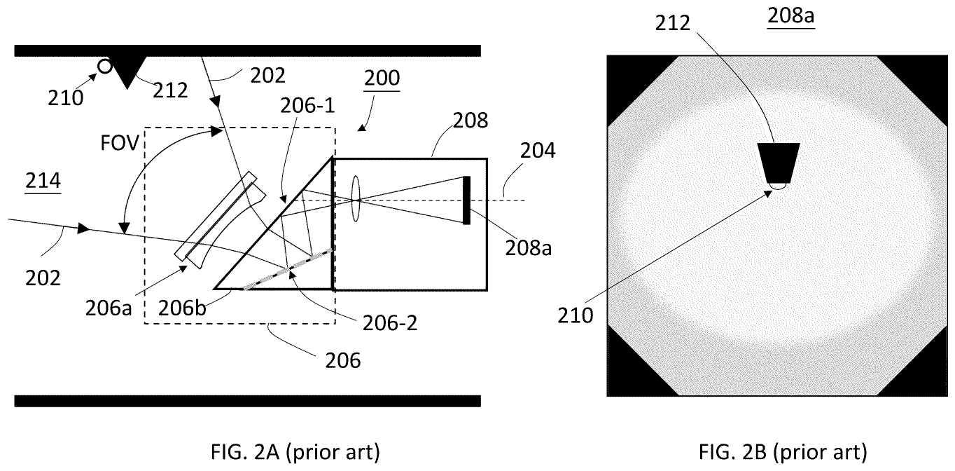

### DETAILED DESCRIPTION

Some preferred embodiments of the present invention will now be described in greater detail. However, it should be recognized that the preferred embodiments of the present invention are provided for illustration rather than limiting the present invention. In addition, the present invention can be practiced in a wide range of other embodiments besides those explicitly described, and the scope of the present invention is not expressly limited except as specified in the accompanying claims.

To overcome the deficiency encountered by the conventional endoscope, the dual view endoscope is proposed. The dual view endoscope includes the functional module in front of the conventional endoscope. The functional module is an optical module which can simultaneously capture both forward-view and side-view images in real-time.

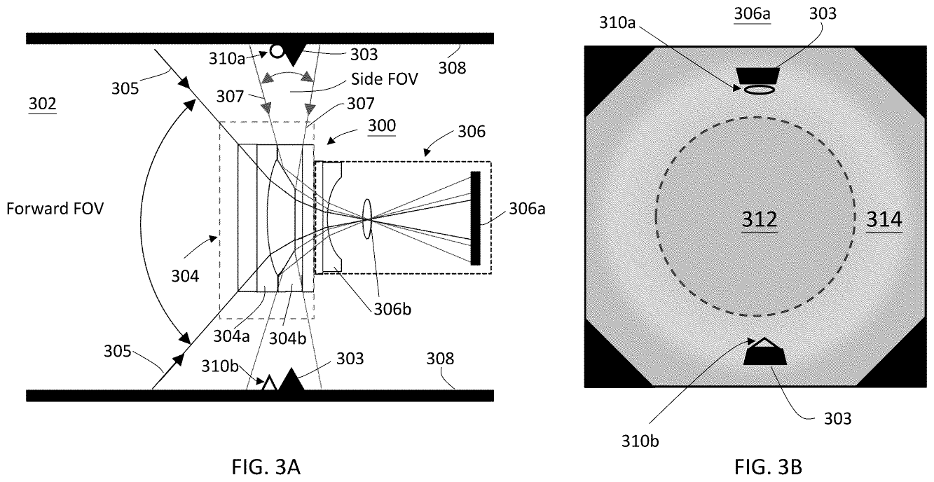

FIG. 3A illustrates a schematic drawing showing a proposed dual view endoscope 300 used to inspect an internal cavity 302 with a bulge structure 303 and objects of interest, for example interested particles (310a, 310b), formed on the bulge structure 303, according to one embodiment of the present invention. The dual view endoscope 300 includes a functional module 304 attached to an image module 306. The functional module 304 includes a meniscus lens 304a and a freeform lens 304b attached to the meniscus lens 304a. The meniscus lens includes two surfaces, one surface has planer shape (planar surface) and the other surface has spherical or aspherical shape (spherical or aspherical surface). The freeform lens 304b includes one surface has planer shape (planar surface) and the other surface has freeform shape (freeform surface). The freeform lens 304b is located between the meniscus lens 304a and the image module 306. The meniscus lens 304a is configured to collect light rays 305 from the forward-viewing direction of the dual view endoscope 300. The freeform lens 304b is rotational symmetric to its optical axis, which is used to act as an annular prism for deflecting light rays 307 from different sides of the sidewall 308 and then entering into an image sensor 306a of the image module 306. The freeform lens 304b is disposed between the meniscus lens 304a and the image module 306, where the meniscus lens 304a is attached to the freeform lens 304a by placing its spherical or aspherical surface adjacent to the freeform surface of the freeform lens 304b. The functional module 304 including the meniscus lens 304a and the freeform lens 304b is attached to or coupled to the image module 306, which can simultaneously capture distal scene and interested particles (310a, 310b) formed on the bulge structure 303 of the internal cavity 302. The image sensor 306a and the functional module 304 are aligned with an axial direction (not shown) of the dual view endoscope 300. In some embodiments, the image module 306 may include a lens set 306b and an image sensor 306a.

FIG. 3B illustrates an image captured by the image sensor 306a, including distal scene from forward field of view (FOV) and the interest particles (310a, 310b) formed on the bulge structure 303. As shown in FIG. 3B, light rays from the forward-viewing direction, i.e., from forward FOV, which can be collected by the functional module 304 and imaged onto the central part 312 of the image sensor 306a. The light rays from lateral directions, i.e., from side FOVs, including light rays reflected from the bulge structure 303 and the interested particles (310a, 310b) formed on the bulge structure 303 at different sides of the sidewall 308 can be deflected by the freeform lens 304b and imaged on to an annular region 314 in vicinity of the central part 312 of the image sensor 306a. In some embodiments, the image sensor 306a may include a complementary metal oxide semiconductor (CMOS) image sensor, a charge coupled device (CCD) image sensor, or the likes.

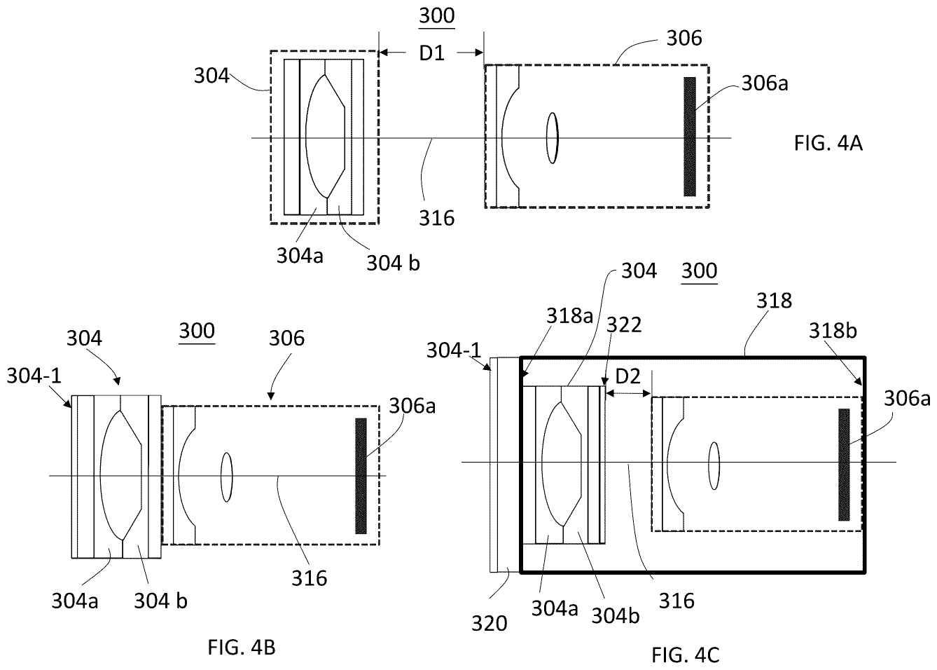

Details of the functional module 304 and how it coupled to the image module 306 will be discussed in the following FIGS. 4-6 and their accompanying descriptions.

FIGS. 4A-4C illustrate various embodiments of the functional module 304 coupled to the image module 306 to act as the dual view endoscope 300 proposed by the present invention.

FIG. 4A illustrates the functional module 304 coupled to the image module 306 according to one embodiment of the present invention. The functional module 304, including the meniscus lens 304a and the freeform lens 304b attached to the meniscus lens 304a, is coupled in front of the image module 306. The freeform lens 304b is disposed between the meniscus lens 304a and the image sensor 306a of the image module 306 while the meniscus lens 304a is attached to the freeform lens 304b by placing its spherical or aspherical surface adjacent to the freeform surface of the freeform lens 304b. The image sensor 306a and the functional module 304 are aligned with an axial direction 316 of the dual view endoscope 300. In some embodiments, the functional module 304 can be separated with the image module 306 by one predetermined distance D1.

FIG. 4B illustrates another embodiment of the functional module 304 coupled to the image module 306. The functional module 304, including the meniscus lens 304a and the freeform lens 304b attached to the meniscus lens 304a, is directly attached in front of the image module 306. The freeform lens 304b is disposed between the meniscus lens 304a and the image sensor 306a of the image module 306 while the meniscus lens 304a is attached to the freeform lens 304b by placing its spherical or aspherical surface adjacent to the freeform surface of the freeform lens 304b.

In some embodiments, an anti-fogging coating 304-1 can be formed on the top surface of the functional module 304, i.e., the outmost surface of the meniscus lens 304a, to prevent it from fogging.

FIG. 4C illustrates another alternative embodiment of the functional module 304 coupled to the image module 306. The functional module 304, including at least the meniscus lens 304a and the freeform lens 304b attached to the meniscus lens 304a, is coupled to the image module 306 through a housing 318. The functional module 304 and the image module 306 are respectively attach to the first inner surface 318a of the housing 318 and the second inner surface 318b of the housing 318 facing the first inner surface 318a, both the functional module 304 and the image module 306 are disposed inside the housing 318. The functional module 304 including the meniscus lens 304a and the freeform lens 304b attached to the meniscus lens 304a, where the freeform lens 304b is disposed between the meniscus lens 304a and the image module 306. The meniscus lens 304a is attached to the freeform lens 304b by placing its spherical surface or aspherical surface adjacent to the freeform surface of the freeform lens 304b. The functional module 304 and the image sensor 306a of the image module 306 are aligned with an axial direction 316 of the dual view endoscope 300. In some embodiments, an outmost cover glass 320 is disposed in front of the functional module 304 and attached to an outer surface of the housing 318. In some embodiments, the functional module 304 and the image module 306 are separated by another predetermined distance D2. In some embodiments, the anti-fogging coating 304-1 is formed on a top surface of the outmost cover glass 320. In order to have long-lasting, or permanent anti-fog performance, the anti-fogging coating 304-1 is typically formulated with large amounts of surfactants in combination with hydrophilic networks. The anti-fogging coating 304-1 can be obtained by coating the glass surface with anti-fog coating material that is hydrophilic, moisture-absorptive, insoluble and excellent in surface hardness. In some embodiments, the anti-fog coating material contains a polyacrylic acid compound, polyvinyl alcohol and acetylacetone, and optionally sodium silicate. In some embodiments, an anti-reflective coating 322 is formed on rear surface of the functional module 304, i.e., the planar surface of the freeform lens 304b of the functional module 304. In some embodiments, the housing 318 may include light-transmitting material for the light rays from the forward-viewing direction and lateral directions being able to pass through.

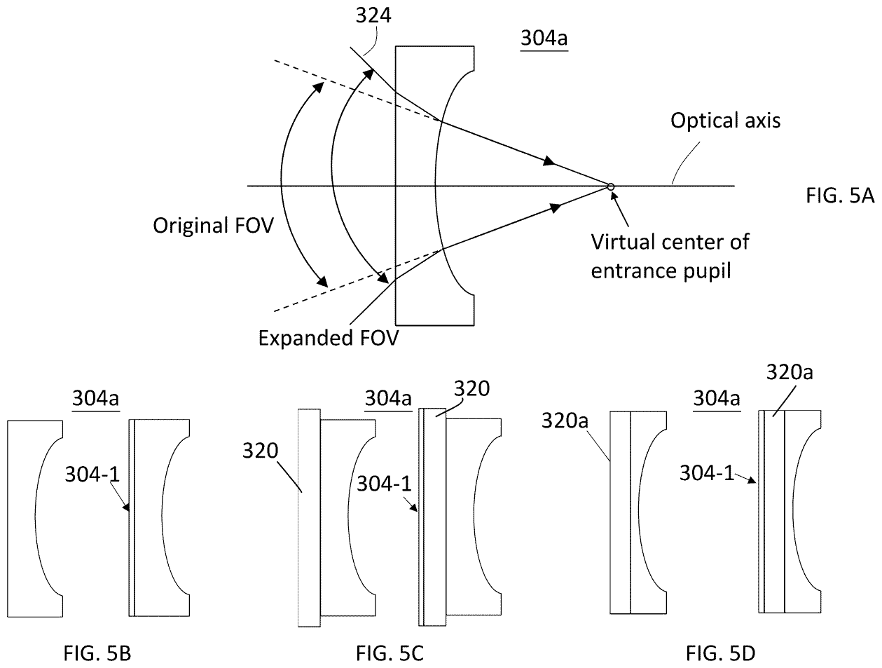

FIG. 5A illustrates a cross-sectional view of the meniscus lens 304a of the functional module 304 according to one embodiment of the present invention. The meniscus lens 304a has spherical or aspherical shape on one side and it has negative refractive power, and the original field of view (FOV) of the forward-viewing direction of the image module 306 can be expanded after placing the meniscus lens 304a in front of the image module 306 (referring to FIGS. 3-4). In FIG. 5A, the angle expands by dashed lines represents the original FOV of the image sensor 306a of the image module 306 (referring to FIGS. 3-4) without placing the functional module 304 and the incoming chief rays 324 can be deflected by adding the meniscus lens 304a, the meniscus lens 304a with spherical or aspherical surface can result an expanded FOV. Generally, the chief ray refers to the light ray from an off-axis point in the object passing through the center of an aperture. In this case, the chief rays 324 pass the virtual center of entrance pupil as indicated in FIG. 5A.

FIGS. 5B-5D illustrate various embodiments of the meniscus lens 304a of the present invention.

Referring to FIGS. 5B-5C, the meniscus lens 304a of the functional module 304 can be directly fabricated by injection molding. The molding lens might be coated with the anti-fogging coating 304-1 on the flat side (planar surface) of the meniscus lens 304a, as shown in the right figure of FIG. 5B, for preventing or inhibiting the buildup of condensation on the lens surface. Referring to FIG. 5C, the molding lens might be attached to a cover glass 320 to act as a protection window (left figure). The cover glass 320 can be coated with the anti-fogging coating 304-1, as shown in the right figure of FIG. 5C, for preventing or inhibiting the buildup of condensation on the lens surface. In some embodiments, the material of meniscus lens 304a can be glass, quartz or plastic.

Referring to FIG. 5D, the meniscus lens 304a of the functional module 304 proposed by the present invention can be polymer and can be fabricated by lens replication on a glass substrate 320a (left figure). The lens replication can be accomplished by dispensing lens material, such as UV curable or thermal resin, over the glass substrate; in the proceeding step providing a lens mold with the meniscus lens profile shape to replicate the meniscus lens 304a and then curing to solidify the meniscus lens 304a. The glass substrate 320a might be coated with an anti-fogging coating 304-1 (right figure) for preventing or inhibiting the buildup of condensation on the lens surface.

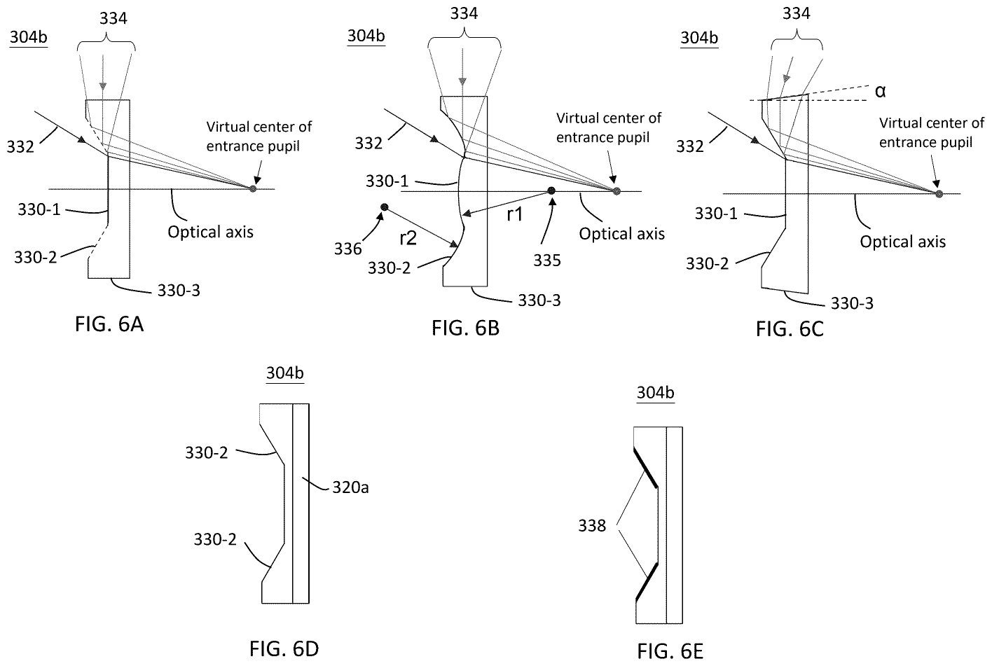

FIGS. 6A-6E illustrate various embodiments of the freeform lens 304b of the functional module 304 disclosed by the present invention.

FIG. 6A illustrates a cross-sectional view of the freeform lens 304b of the functional module 304 according to one embodiment of the present invention, which is rotational symmetric to its optical axis. The freeform lens 304b consists three surface regions, i.e., a central surface region 330-1, a distal surface region 330-2 and a circumferential region 330-3, and each surface region has its unique shape. The distal surface region 330-2 is extended from the central surface region 330-1 and the circumferential region 330-3 located on an outer edge of the freeform lens 304b, where the circumferential region 330-3 encloses the distal surface region 330-2 and the central surface region 330-1. The light rays enter the aforementioned meniscus lens 304a (referring to FIGS. 3-4) and pass the central surface region 330-1, for example chief ray 332 coming from the forward-viewing direction (distal end) of the dual view endoscope 300, which is associated with the forward FOV, enabling that the distal scene is imaged onto the central part 312 of the image sensor 306a (referring FIG. 3B). In this case, the chief ray 332 passes through the virtual center of entrance pupil. The light rays entered the circumferential region 330-3 are reflected by total internal reflection in the distal surface region 330-2, for example chief rays 334 coming from lateral directions, which are associated with side FOVs, enabling that the scene of the sidewall is imaged onto the annular region 314 in vicinity of the central part 312 of the image sensor 306a (referring to FIG. 3B). The chief rays 334 coming from the lateral directions also pass through the virtual center of entrance pupil.

FIG. 6B illustrates a cross-sectional view of the freeform lens 304b of the functional module 304 according to other embodiment of the present invention, which is rotational symmetric to its optical axis. The freeform lens 304b consists three surface regions, i.e., the central surface region 330-1, the distal surface region 330-2 and the circumferential region 330-3, where each surface region has its radius of curvature. The central surface region 330-1 has a radius of curvature r1 and its corresponding center of curvature 335. The distal surface region 330-2 has a radius of curvature r2 and its corresponding center of curvature 336, and the circumferential region 330-3 has infinite radius of curvature. Other details regarding this embodiment that are similar to those for the previously described embodiment (referring to FIG. 6A) will not be repeated herein. As shown in FIG. 6B, the curved surface of the distal surface region 330-2 can expand the side FOVs.

FIG. 6C illustrates a cross-sectional view of a freeform lens 304b of the functional module 304 according to another embodiment of the present invention, which is rotational symmetric to its optical axis. The freeform lens 304b consists three surface regions, i.e., the central surface region 330-1, the distal surface region 330-2 and the circumferential region 330-3, and each region has its unique shape. Particularly, a wedge structure is formed on the circumferential region 330-3 and has a corresponding wedge angle α with respected to its symmetric axis (optical axis), i.e. the wedge structure is defined by the wedge angle α. Other details regarding this embodiment that are similar to those for the previously described embodiment (referring to FIG. 6A) will not be repeated herein. Details of light rays regarding this embodiment that are similar to those for the previously described embodiment will not be repeated herein. As the light rays enter the circumferential region 330-3 that includes the wedge structure with the corresponding wedge angle α, the side FOVs are depended on the wedge angle α. As a result, the side FOVs can be steered by varying the wedge angle x. In some embodiments, the freeform lens mentioned in FIGS. 6A-6C can be fabricated by injection molding. In some embodiments, the material of freeform lens can be glass, quartz or plastic.

Referring to FIGS. 6D-6E, the freeform lens 304b of the functional module 304 can be polymers and can be fabricated by lens replication on the glass substrate 320a. The lens replication can be accomplished by dispensing lens material, such as UV curable or thermal resin, over the glass substrate; in the proceeding step providing a lens mold with the freeform lens profile shape to replicate the freeform lens 304b and then curing to solidify the freeform lens 304b. The distal surface region 330-2 of the freeform lens 304b might be coated with a reflective layer 338 (FIG. 6E) for reflecting light rays coming from the lateral directions into the image sensor 306a (referring to FIGS. 3-4).

The dual view endoscope of the present invention has the following advantages: (1). the dual view endoscope simultaneously captures both forward-view and side-view images in real-time; (2). assembly of the dual view endoscope is easily accomplished by integrating the functional module with an existing endoscope; (3). cost of the rotationally symmetric freeform lens is lower than the state-of-art solution; (4). the freeform lens provides side FOV functionality, which can be fabricated by injection molding and lens replication through wafer level optics technology for mass production.

While various embodiments of the present invention have been described above, it should be understood that they have been presented by a way of example and not limitation. Numerous modifications and variations within the scope of the invention are possible. The present invention should only be defined in accordance with the following claims.

What is claimed is:

1. A dual view endoscope, comprising:
an image sensor; and
a functional module disposed in front of said image sensor, said functional module including at least a first lens and a second lens attached to said first lens, said second lens being located between said first lens and said image sensor;
wherein said first lens is configured to expand field of view of said image sensor, said second lens being configured to collect light rays from lateral directions and deflect said light rays into said image sensor;
wherein said image sensor and said functional module are aligned with an axial direction of said dual view endoscope.

2. The endoscope of claim 1, wherein a lens module is disposed between said functional module and said image sensor to collect said light rays from a forward-viewing direction of said dual view endoscope.

3. The endoscope of claim 2, wherein said functional module includes a first anti-fogging coating formed on a first surface far away from the said second lens.

4. The endoscope of claim 2, wherein said first lens is a meniscus lens, wherein said first lens includes a spherical or aspherical shape on one side that is placed adjacent to said second lens.

5. The endoscope of claim 2, wherein said first lens includes a negative refractive power.

6. The endoscope of claim 2, further comprising a cover glass formed adjacent to said first lens.

7. The endoscope of claim 2, wherein said first lens is formed by lens replication on a glass substrate having a second anti-fogging coating.

8. The endoscope of claim 2, wherein said second lens is a freeform lens, wherein said second lens is rotational symmetric to its optical axis and consists three surface regions being placed adjacent to said first lens, each surface region of said second lens having a radius of curvature.

9. The endoscope of claim 8, wherein said surface regions of said second lens includes:
a central surface region located on a central portion of said second lens;
a distal surface region extended from said central surface region; and
a circumferential region located on an outer edge of said second lens and enclosed said distal surface region and said central surface region.

10. The endoscope of claim 9, wherein said light rays entered said first lens and passed said central surface region are imaged onto a central part of said image sensor.

11. The endoscope of claim 10, wherein said light rays from said lateral directions entered said circumferential region and reflected by said distal surface region are imaged onto an annular region in vicinity of said central part of said image sensor.

12. A lens module for endoscope, comprising:
a first lens;
a second lens attached to said first lens, said second lens being located between said first lens and said endoscope;
wherein said first lens is configured to expand field of view of said endoscope, said second lens being configured to collect light rays from lateral directions and deflect said light rays onto an image sensor of said endoscope.

13. The lens module of claim 12, wherein said first lens includes spherical or aspherical shape on one side that is placed adjacent to said second lens.

14. The lens module of claim 12, wherein said first lens includes negative refractive power to expand said field of view of said endoscope in its forward-viewing direction.

15. The lens module of claim 12, wherein said first lens is formed by lens replication on a glass substrate having an anti-fogging coating.

16. The lens module of claim 12, wherein said second lens is rotational symmetric to its optical axis and consists three surface regions being placed adjacent to said first lens, each surface region of said second lens having a radius of curvature.

17. The lens module of claim 16, wherein said surface regions of said second lens includes:
a central surface region located on a central portion of said second lens;
a distal surface region extended from said central surface region; and
and a circumferential region located on an outer edge of said second lens and enclosed said distal surface region and said central surface region.

18. The lens module of claim 17, wherein said light rays entered said first lens and passed said central surface region are imaged onto a central part of said image sensor.

19. The lens module of claim 18, wherein said light rays from said lateral directions entered said circumferential region and reflected by said distal surface region are imaged onto an annular region in vicinity of said central part of said image sensor.

20. The lens module of claim 16, wherein said second lens includes an anti-reflecting coating formed on a surface far away from said first lens.
